(19) United States
(12) Patent Application Publication
(10) Pub. No.: US 2021/0156931 A1
(43) Pub. Date: May 27, 2021

(54) MAGNETIC FIELD MEASURING APPARATUS

(71) Applicant: Takashi YASUI, Tokyo (JP)

(72) Inventor: Takashi YASUI, Tokyo (JP)

(73) Assignee: Ricoh Company, Ltd., Tokyo (JP)

(21) Appl. No.: 16/953,824

(22) Filed: Nov. 20, 2020

(30) Foreign Application Priority Data
Nov. 25, 2019 (JP) 2019-212345

(51) Int. Cl. G01R 33/035; H01L 39/02

(52) U.S. Cl. CPC G01R 33/0356; H01L 39/045; H01L 39/025

(57) ABSTRACT

A magnetic field measuring apparatus that includes a first board having a plurality of first connecting parts, at least one second board connected to the plurality of first connection parts, and a power supply unit supplying power to the first board and the at least one second board so as to measure a magnetic field includes at least one first voltage regulator disposed on the first board to generate a first voltage using power from the power supply unit; and at least one second voltage regulator disposed on any one of the at least one second board to generate a second voltage using the first voltage.

### CROSS-REFERENCE TO RELATED APPLICATIONS

The present application claims priority under 35 U.S.C. § 119 to Japanese Patent Application No. 2019-212345, filed Nov. 25, 2019, the contents of which are incorporated herein by reference in their entirety.

### BACKGROUND OF THE INVENTION

### Field of the Invention

The present invention relates to magnetic field measuring apparatus.

### Description of the Related Art

In biomagnetic field measurements using superconducting quantum interference devices (SQUIDS), which are superconducting rings with Josephson junctions, the measurement characteristics are nonlinear. Therefore, in a magnetic field measuring apparatus having superconducting quantum interference devices, linearization is performed using a FLL (Flux Locked Loop) circuit to measure the magnetic field. Hereinafter, superconducting quantum interference devices are also referred to as SQUIDs.

There are two types in FLL circuits: an analog FLL method, which is made exclusively of analog circuits, and a digital FLL method, which is made of circuits that are once made digital and then made analog again. In the biomagnetic field measurement, because multiple channels are commonly used, the digital FLL method has been frequently used because of a reduction of variation between channels, a reduction of system costs, and an ease of data processing, as well as an advance in semiconductor technology.

Because the biological magnetic field signals are very weak compared to noises such as an environmental magnetic field, it is necessary to divide the magnetic field signals measured by the SQUID into components of the biological magnetic field signals and the noises in the measurement of the magnetic field by using a magnetic field measuring apparatus. For example, the magnetic field signal detected by the SQUID is decomposed into independent components of each current source and other sources of biological activity, and it is determined whether each independent component is a noise component.

Then, the biological magnetic field signal is restored by removing the independent component judged as the noise component (see Japanese Patent No. 4236352).

The magnetic field measuring apparatus having the digital FLL circuit has been applied to an independent application such as a spinal magnetometer (MSG: Magnetospinograph), cardio magnetometer (MCG: Magnetocardiograph), or brain magnetometer (MEG: Magnetoencephalograph). This is because the characteristics of the biological magnetic field signals (signal size, bandwidth, and the number of channels required for measurement) differ depending on the measurement target. When a magnetic field measuring apparatus is applied to multiple applications such as MSG, MCG, and MEG, a circuit or system configuration is required to efficiently process differences in the properties of different biomagnetic signals depending on a measurement target portion.

In recent years, the number of channels (the number of SQUIDs) in the magnetic field measuring apparatuses are prone to increase in order to improve the measurement accuracies. The number of channels also increases when the magnetic field measuring apparatus is applied in a variety of applications. As the number of channels increases, the size of the processing circuit that processes the magnetic field signals measured in the SQUID increases, and the current consumption increases. Power source noises increase as current consumptions increase. Because the biomagnetic field signals are very weak, a mechanism to suppress the power source noises are necessary in order not to lower measurement accuracies.

The disclosed technique has been developed in view of the above problems and is intended to reduce noises contained in a second voltage generated on the basis of power supplied from a power supply and to improve the measurement accuracy.

### SUMMARY OF THE INVENTION

In order to solve the above-described technical problem, a magnetic field measuring apparatus of the present invention includes a first board having a plurality of first connections; at least one or more second boards connected to the first connection; and a power supply providing power to the first board and the second board, wherein the magnetic field measuring apparatus comprises: at least one first voltage regulator provided on the first board to generate a first voltage using power from the power supply; and at least one second voltage regulator provided on at least one of the second boards to generate a second voltage using the first voltage.

### BRIEF DESCRIPTION OF THE DRAWINGS

FIG. 1 is a block diagram illustrating an example of a magnetic field measuring apparatus according to a first embodiment of the present invention.

FIG. 2 is a block diagram illustrating an example of a supply path of a power source voltage of the magnetic field measuring apparatus illustrated in FIG. 1.

FIG. 3 is a block diagram illustrating an example of the magnetic field measuring apparatus according to a second embodiment of the present invention.

FIG. 4 is a block diagram illustrating an example of a backplane unit illustrated in FIG. 3.

FIG. 5 is a diagram illustrating an example of power source noise generated when using a post-regulator.

FIG. 6 illustrates an example of a board connected to the backplane unit in the magnetic field measuring apparatus illustrated in FIG. 3.

FIG. 7 illustrates another example of the board connected to the backplane unit in the magnetic field measuring apparatus illustrated in FIG. 3.

FIG. 8 illustrates another example of the board connected to the backplane unit in the magnetic field measuring apparatus illustrated in FIG. 3.

FIG. 9 illustrates another example of the board connected to the backplane unit in the magnetic field measuring apparatus illustrated in FIG. 3.

FIG. 10 is a block diagram illustrating an example of a magnetic field measuring apparatus according to a third embodiment of the present invention.

### DESCRIPTION OF THE EMBODIMENTS

Hereinafter, embodiments will be described with reference to the drawings. In each drawing, the same component is indicated by the same reference numeral and an overlapping description may be omitted.

### First Embodiment

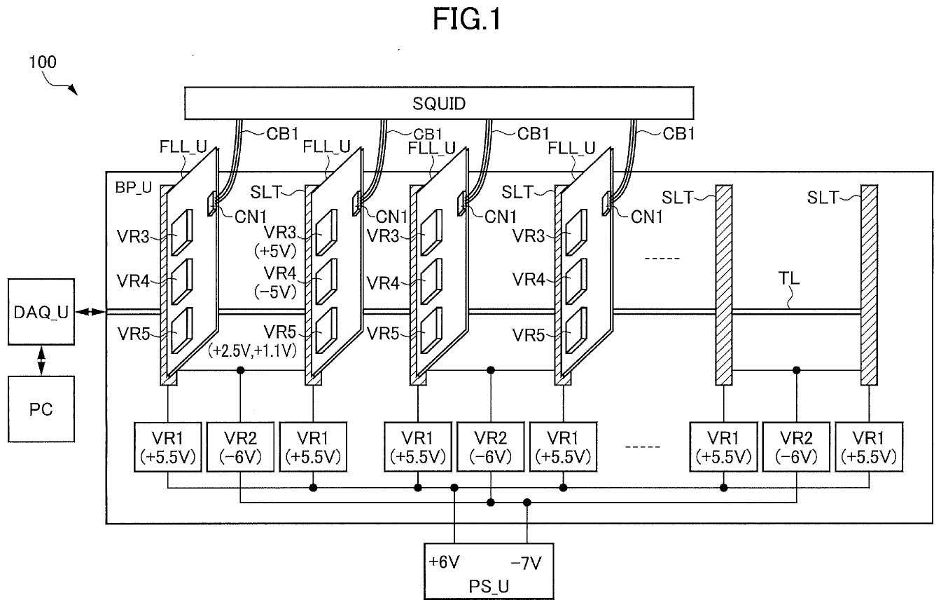

FIG. 1 is a block diagram illustrates an example of a magnetic field measuring apparatus according to a first embodiment of the present invention. For example, the magnetic field measuring apparatus 100 (biomagnetic field measuring apparatus) illustrated in FIG. 1 employs a digital FLL method and is applicable to a spinal magnetometer, cardio magnetometer, and brain magnetometer. The magnetic field measuring apparatus 100 illustrated in FIG. 1 may also be applied to the measurement of a nerve magnetic field or muscle magnetic field.

TABLE 1

| | SPINAL | CARDIO | BRAIN |
|---|---|---|---|
| | MAGNE- | MAGNE- | MAGNE- |
| | TOMETER | TOMETER | TOMETER |
| | (MSG) | (MCG) | (MEG) |
| MAGNETIC | SEVERAL~ | SEVERAL | 10 f~10 p |
| SENSITIVITY | SEVERAL 10 f | 10 f~100 p | |
| (T) | | | |
| SIGNAL | 100~SEVERAL k | 0~1k | 0(0.1)~ |
| BAND (Hz) | | | SEVERAL 100 |
| NUMBER OF | ~128 | 1~32~128 | 128~306 |
| CHANNELS | | | |

Table 1 illustrates an example of the magnetic sensitivity (T), signal band (Hz), and number of channels for the measurement of the biomagnetic field signals for each intended usage (measurement target). As illustrated in Table 1, the magnetic sensitivity, signal band, and number of channels required for measuring the biomagnetic field differ among the spinal magnetometer (MSG), cardio magnetometer (MCG), and brain magnetometer (MEG). The required signal band varies for each of the spinal magnetometer, cardio magnetometer, and brain magnetometer, so the sampling frequency covering the signal band also varies. For example, the sampling frequency for each intended usage may be several to ten times the signal band.

The spinal magnetometer receives electrical stimulation from the outside and measures the biomagnetic field induced by the electrical stimulation. An artifact (noise) caused by the electrical stimulation influences a measurement result. Generally, the artifact is larger than the biomagnetic field, so a wide dynamic range is required.

In measuring the magnetic field, not only a sampling frequency used in the spinal magnetometer, cardio magnetometer, and brain magnetometer but also the sampling frequency used in a long-term mode, environmental magnetic field measurement, and the like is required. The long-term mode is used, for example, in the diagnosis of epilepsy, to measure the biomagnetic field with a brain magnetometer in the presence of stimuli such as sound, video, or electricity to a subject. In the environmental magnetic field measurement, by measuring the magnetic field of the surrounding environment of the magnetic field measuring apparatus 100 without measuring the biomagnetic field of the subject, it is confirmed that the magnetic field measuring apparatus 100 is operating normally, and it is judged whether the biomagnetic field can be measured.

In addition, data synchronized with a conventional electroencephalograph (EEG) or electrocardiograph (ECG) may be necessary for diagnosis as basic confirmation of operation in the cardio magnetometer or brain magnetometer. In the spinal magnetometer, data synchronized with the electromyograph (EMG) or electrocardiograph may be necessary for diagnosis. Because the electroencephalograph, electrocardiograph, and electromyograph are individual devices, the synchronization of the data between these devices and the magnetic field measuring apparatus 100 is required for each device.

The magnetic field measuring apparatus 100 includes a power source unit PS_U, a backplane unit BP_U, a data processing unit FLL_U, a SQUID, a data acquisition unit DAQ_U, and a PC (Personal Computer). The PC may not be included in the magnetic field measuring apparatus 100, and a server may be used instead of the PC. In the example illustrated in FIG. 1, the power source unit PS_U generates DC (Direct Current) voltages of +6V and −7V. The power source unit PS_U is an example of a power supply unit, and a data acquisition unit DAQ_U is an example of a data acquisition unit.

The backplane unit BP_U, for example, is formed like a printed wiring board including a plurality of connector slots SLT in which a board such as the data processing unit FLL_U can be connected, and a plurality of voltage regulators VR1 and VR2. The backplane unit BP_U is an example of a first board, and the connector slot SLT is an example of a first connecting part. Voltage regulators VR1 and VR2 are examples of a first voltage regulator, and the voltages respectively generated by the voltage regulators VR1 and VR2 are examples of a first voltage.

The backplane unit BP_U is mounted in a housing (not illustrated) of the magnetic field measuring apparatus 100 and is integral with the housing. By mounting the voltage regulators VR1 and VR2 in the backplane unit BP_U, an extra board mounting the voltage regulators VR1 and VR2 is not required to be installed in the housing. Therefore, it is possible to improve a storage efficiency of the parts in the housing. In addition, because the voltage regulators VR1 and VR2 can be directly connected to a power source line wired on the backplane unit BP_U, the routing of the power source line can be minimized and the influence of exogenous noise on the power source line can be minimized.

In the example illustrated in FIG. 1, a voltage regulator VR1 is provided for each connector slot SLT, receives +6 V supplied from the power source unit PS_U, generates +5.5 V, and outputs the generated +5.5 V to the corresponding connector slot SLT. The voltage regulator VR2 is provided for each of the two connector slots SLT and generates −6V when receiving the −7V supplied from the power source unit PS_U and outputs the generated −6V to the corresponding connector slot SLT. The number of voltage regulators VR1 and VR2 mounted in the backplane unit BP_U is determined in accordance with the current consumed by the data processing unit FLL_U.

Each connector slot SLT is connected to each other via a common transmission line TL (wiring pattern) formed on the backplane unit BP_U, and the transmission line TL is connected to the data acquisition unit DAQ_U via a connection (second connecting part) not illustrated. Transmission methods such as a data signal or the like using the transmission line TL may be by a serial transmission through a serial interface or a parallel transmission through a parallel interface. A transmission line TL is an example of a common signal line.

By providing a common transmission line TL, the magnetic field data generated by the data processing unit FLL_U connected to any connector slot SLT can be transmitted to the data acquisition unit DAQ_U according to a common interface specification. In other words, when a circuit board connected to the connector slot SLT and extending the function of the magnetic field measuring apparatus 100 is newly designed, data can be acquired by the data acquisition unit DAQ_U by designing the circuit board together with an interface specification.

Each data processing unit FLL_U is formed like a printed wiring board, for example, having a connector CN1 connecting a plurality of voltage regulators VR3, VR4, and VR5 and a cable CB1 used for connecting the SQUID. Each data processing unit FLL_U is attachably and detachably connected to any connector slot SLT of the backplane unit BP_U. The data processing unit FLL_U is an example of a second board. The voltage regulators VR3, VR4, and VR5 are examples of a second voltage regulator, and the voltages generated by the voltage regulators VR3, VR4, and VR5 are examples of a second voltage.

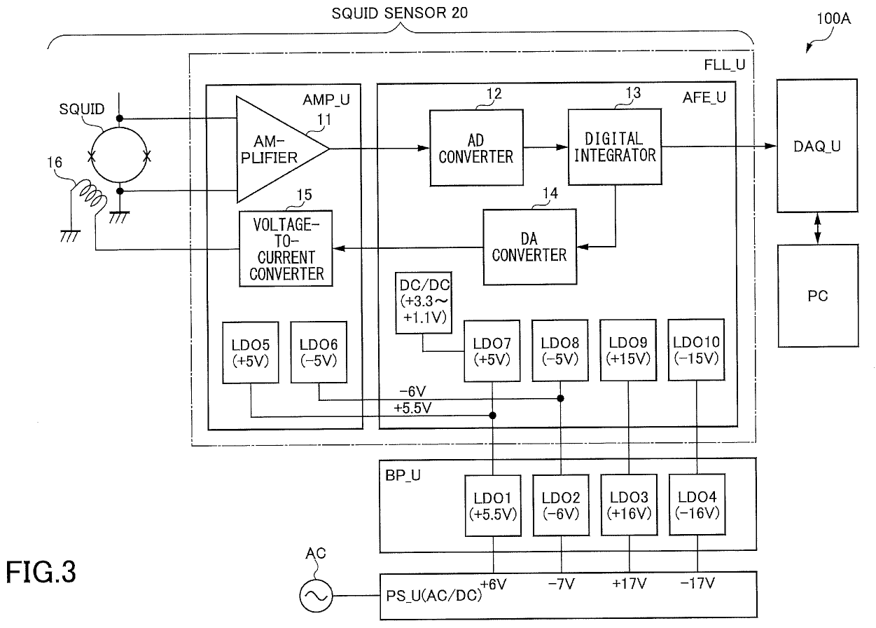

The SQUID illustrated in FIG. 1 is shaped like a rectangular, which includes a plurality of SQUIDs, and each data processing unit FLL_U is connected to a predetermined number (for example, 16 SQUIDs). That is, each data processing unit FLL_U has 16 channels. The operation of the data processing unit FLL_U will be illustrated in FIG. 3 and thereafter. Hereinafter, the voltage regulators VR1-VR5 are referred to as voltage regulators VR when they are described without differentiations. For example, the voltage regulator VR may be a switching regulator or the like and an LDO (Low DropOut) regulator may be used therein.

The voltage regulator VR3 receives +5.5 V generated by the voltage regulator VR1 and generates +5 V and supplies the generated voltage to a circuit in the data processing unit FLL_U. The voltage regulator VR4 receives −6V generated by the voltage regulator VR2 and generates −5V and supplies the generated voltage to a circuit in the data processing unit FLL_U. The voltage regulator VR3 receives +5.5 V generated by the voltage regulator VR1 and generates +2.5 V and +1.1 V and supplies the generated voltage to a circuit in the data processing unit FLL_U. The voltage generated in the data processing unit FLL_U (+5V, −5V, +2.5V, +1.1V) is used only in the circuit in the data processing unit FLL_U.

The data acquisition unit DAQ_U receives the magnetic field data (measured data) measured by the SQUID and generated in the processing by the data processing unit FLL_U through the transmission line TL and performs filtering and thinning processing of the received magnetic field data to be transferred to the PC. The PC saves the magnetic field data to a hard disk device, etc., and displays a waveform, etc. indicating the stored magnetic field data on the display device.

The value of the voltage output by the power source unit PS_U and the value of the voltage output by the voltage regulators VR1 to VR5 are examples, and the voltage value is not limited thereto. Further, the number of data processing units FLL_U connected to the backplane unit BP_U is not limited to four, and any number of data processing units FLL_U corresponding to the required number of channels can be connected. The number of voltage regulators VR3, VR4, and VR5 mounted in each data processing unit FLL_U is not limited to one.

In addition to the data processing unit FLL_U, other circuit boards can be connected to each connector slot SLT. When a new circuit board is designed to connect to the connector slot SLT, the circuit board is mounted with a second voltage regulator VR connected in series to at least one of the voltage regulators VR1, VR2. Accordingly, it is possible to have a two-stage structure of post-regulator which is a voltage regulator connected to the power source unit PS_U, and it is possible to reduce the power source noise contained in the power supply supplied to the processing circuit mounted on the newly designed circuit board.

The second voltage regulator VR mounted on the circuit board has the capability of matching the current consumption of the processing circuit mounted on the circuit board and can have a predetermined power supply rejection ratio (PSRR: Power Supply Rejection Ratio). Therefore, when the power source noise is suppressed by the two-stage structure of post-regulator, the power source noise reduction amount can be set to a desired amount by combining the PSRR of the voltage regulators VR1 and VR2 and the second voltage regulator VR according to the current characteristics of the circuit board. As a result, the power source noise of the circuit board connected to the connector slot SLT can be suppressed, and the measurement accuracy of the magnetic field measuring apparatus 100 can be prevented from being deteriorated.

Because a biological signal, especially a magnetic field signal, is generally weak and susceptible to noise, circuits that process biological signals are required to have good noise characteristics. In order to mitigate the influence of environmental noise, the magnetic field measuring apparatus 100 is installed in a magnetic shielding room that shields the magnetic field, and a cooling device for the SQUID is installed inside the magnetic shielding room. The magnetic shielding room and cooling system are very expensive. Therefore, if the intended usage of the magnetic field measuring apparatus 100 is only either a spinal magnetometer, a cardio magnetometer, or a brain magnetometer, three types of magnetic field measuring apparatuses 100 need to be developed and installed in each of the three magnetic shielding rooms. In this case, the total cost of the magnetic field measuring system becomes very large.

In this embodiment, for example, the number of data processing units FLL_U connected to the backplane unit BP_U can be freely changed. Alternatively, the type of the data processing unit FLL_U connected to the backplane unit BP_U can be changed freely, and a circuit board other than the data processing unit FLL_U can be connected to the backplane unit BP_U. Thus, the magnetic field measuring apparatus 100 placed in one magnetic shield room can function as the spinal magnetometer, cardio magnetometer, or brain magnetometer, thereby significantly reducing the total cost of the magnetic field measuring system.

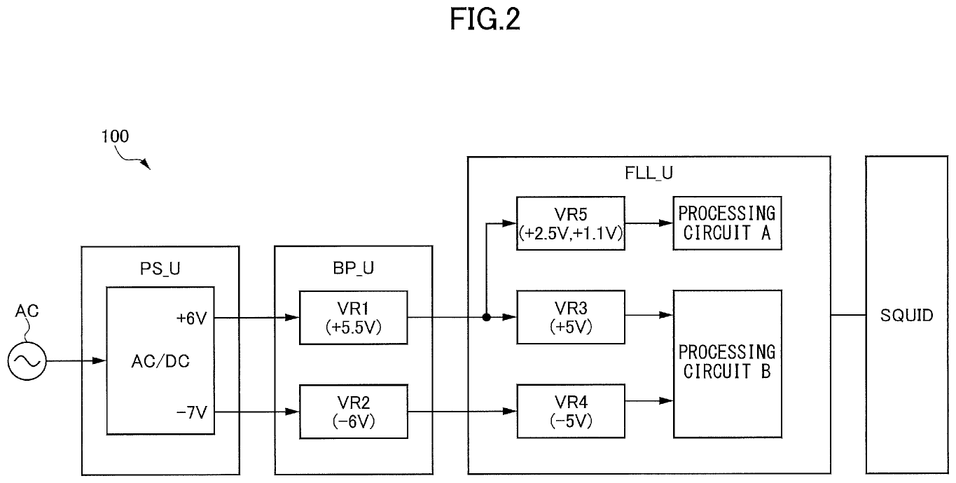

FIG. 2 is a block diagram illustrating an example of the supply path of the power source voltage of the magnetic field measuring apparatus 100 of FIG. 1. As illustrated in FIG. 2, the magnetic field measuring apparatus 100 has a two-stage structure in which at least any one of the voltage regulators VR1 and VR2 mounted in the backplane unit BP_U and at least any one of the voltage regulators VR3 to VR5 mounted in the data processing unit FLL_U are connected in series. Specifically, the voltage regulators VR1 and VR3 are connected in series, the voltage regulators VR1 and VR5 are connected in series, and the voltage regulators VR2 and VR4 are connected in series.

Each voltage regulator VR has a predetermined power source voltage variation removal ratio (i.e., PSRR: Power Supply Rejection Ratio) and can remove noise components contained in the input voltage at a predetermined ratio. For example, the voltage regulators VR3, VR4, and VR5 of the second stage receive a power source voltage in which the noise is suppressed by the voltage regulators VR1 and VR2 of the first stage, further remove noise, and supply the noise-removed power source voltage to a processing circuit A or a processing circuit B in a data processing unit FLL_U. The power source voltages output by the voltage regulators VR3, VR4, and VR5 are supplied only to the processing circuits A and B in the board of the data processing unit FLL_U, and are not supplied to the outside by cables or the like connected to the board of the data processing unit FLL_U. For this reason, it is possible to prevent noise from entering the power source voltage output by the voltage regulators VR3, VR4, and VR5 from the outside via a cable or the like.

In addition, the voltage regulators VR3, VR4, and VR5 are disposed near the processing circuits A and B operated by the power source voltages output by the voltage regulators VR3, VR4, and VR5, so that the noise included in the power source voltages supplied to the processing circuits A and B can be minimized. Accordingly, by making the voltage regulator VR the two-stage structure, it is possible to suppress the precision of the magnetic field measurement by the magnetic field measuring apparatus 100 from being reduced due to the noise. The number of stages of the voltage regulator VR connected in series is not limited to two stages, but may be three or more stages.

As described above, in this embodiment, by connecting a plurality of voltage regulators VR in series, the power source noise supplied from the power source unit PS_U to the processing circuits A and B in the data processing unit FLL_U through the backplane unit BP_U can be reduced. Accordingly, the processing circuits A and B can be operated so as to minimize the influence of power source noise, and the accuracy of the magnetic field measurement by the magnetic field measuring apparatus 100 can be improved.

The voltage regulators VR1 and VR2 can be connected directly to the power source line wired on the backplane unit BP_U, thereby minimizing the routing of the power source line and reducing the effect of exogenous noise on the power source line.

By mounting the voltage regulators VR3 to VR5 on the data processing unit FLL_U in which the data processing circuitry A and B are mounted, which uses the power source voltage generated by the voltage regulators VR3 to VR5, the noise which enters the power source voltage generated by the voltage regulators VR3 to VR5 can be reduced.

Each connector slot SLT is connected to the data acquisition unit DAQ_U through a common transmission line TL formed on the backplane unit BP_U. This allows the biological data generated by the data processing unit FLL_U connected to any connector slot SLT and other circuit boards to be transmitted to the data acquisition unit DAQ_U according to a common interface specification.

When a circuit board is newly designed, by being mounted the second voltage regulator VR connected in series to at least one of the voltage regulators VR1 and VR2 on the circuit board, the power source noise supplied to the processing circuit mounted on the circuit board can be reduced. In this case, the second voltage regulator VR has the capability of matching the current consumption of the processing circuit mounted on the circuit board and can adopt that have a predetermined PSRR. Accordingly, by combining the voltage regulators VR1 and VR2 with the PSRR of the second voltage regulator VR, the reduction amount in the power source noise can be set to a desired amount, and the measurement accuracy of the magnetic field measuring apparatus 100 can be prevented from being deteriorated.

Any type and any number of circuit boards can be connected to the backplane unit BP_U, allowing one magnetic field measuring apparatus 100 to function as the spinal magnetometer, cardio magnetometer, or brain magnetometer located in one magnetic shield room. As a result, it is possible to significantly reduce the total cost of the magnetic field measuring system.

### Second Embodiment

FIG. 3 is a block diagram illustrating an example of a magnetic field measuring apparatus according to a second embodiment of the present invention. For the same structure as illustrated in FIG. 1, the same reference numerals are given and the detailed descriptions thereof are omitted. The magnetic field measuring apparatus 100A illustrated in FIG. 3 has an SQUID (superconducting quantum interference element) and an SQUID sensor 20. The SQUID and the SQUID sensor 20 illustrated in FIG. 3 represent one channel of the magnetic field measuring apparatus 100A. For example, but not particularly limited, the magnetic field measuring apparatus 100A has tens or hundreds of channels.

The SQUID is a highly sensitive magnetic sensor that detects a magnetic field (magnetic flux) generated from a living body through a superconducting ring having a Josephson junction. For example, the SQUID is formed by providing two Josephson junctions in a superconducting ring.

The SQUID generates a voltage that varies periodically with the change in magnetic flux through the superconducting ring. Therefore, the magnetic flux through the superconducting ring can be obtained by measuring the voltage across both ends of the superconducting ring with a bias current applied to the superconducting ring. Hereinafter, the characteristic of the periodic voltage change generated by the SQUID is also referred to as the Φ-V characteristic.

The SQUID sensor 20 includes a data processing unit FLL_U for processing a magnetic field signal detected by the SQUID and a return coil 16. The data processing unit FLL_U includes an amplifier unit AMP_U and a data generation unit AFE_U, and constitutes a so-called digital FLL circuit.

For example, the amplifier unit AMP_U and the data generation unit AFE_U each have the form of a printed wiring board, but may be mounted on a common board. The amplifier unit AMP_U is an example of the third board, and the data generation unit AFE_U is an example of the second board. For example, one data processing unit FLL_U includes a 16-channel SQUID sensor 20. The return coil 16 positioned proximate to the SQUID is mounted on a board other than the board of the data processing unit FLL_U.

For example, the power source unit PS_U is an AC/DC converter (switching power supply) that generates DC voltages of +6V, −7V, +17V, and −17V using AC voltages supplied from AC power source AC (Alternating Current). The power source unit PS_U may be a DC/DC converter that generates a DC voltage using the DC voltage supplied from the DC power source DC.

The backplane unit BP_U, for example, has the form of a printed wiring board and includes LDO (Low DropOut) 1, LDO2, LDO3, and LDO4. LDO1 to LDO4 are examples of the first voltage regulator, and the voltages generated by LDO1 to LDO4 are examples of the first voltages.

The LDO1 receives +6V supplied from the power source unit PS_U and generates a DC voltage of +5.5 V. The LDO2 receives −7V supplied from the power source unit PS_U and generates a DC voltage of −6 V. The LDO3 receives +17V supplied from the power source unit PS_U and generates a DC voltage of +16V. The LDO4 receives −17V supplied from the power source unit PS_U and generates a DC voltage of −16V.

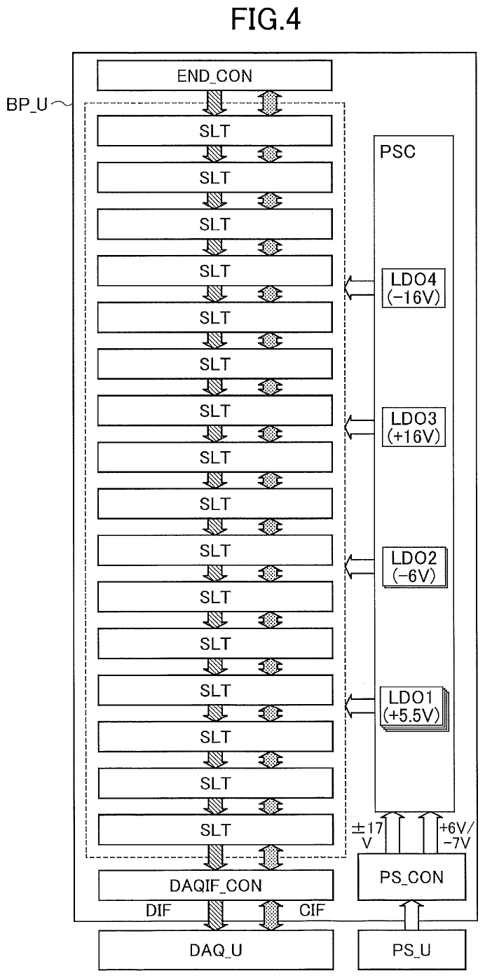

The backplane unit BP_U has a plurality of connector slots SLT (FIG. 4) each for attachably and detachably connecting the boards forming the data generation unit AFE_U. The DC voltages generated by the LDO 1, LDO 2, LDO 3, and LDO 4 are supplied to a circuit mounted on the data generation unit AFE_U connected to the connector slot SLT. The DC voltages (5.5 V, −6 V) output from the LDO1 and LDO2 are also supplied to the amplifier unit AMP_U through the data generation unit AFE_U.

The amplifier unit AMP_U includes an LDO 5, an LDO 6, an amplifier 11, and a voltage-to-current converter 15. The data generation unit AFE_U includes an LDO 7, an LDO 8, an LDO 9, an LDO 10, a DC/DC converter, an AD (Analog-to-Digital) converter 12, a digital integrator 13, and a DA (Digital-to-Analog) converter 14. At least one of the AD converter 12 and the DA converter 14 may be mounted on the amplifier unit AMP_U, and the voltage-to-current converter 15 may be mounted on the data generation unit AFE_U.

The LDO5 and LDO6 are examples of a third voltage regulator, and the voltages generated by the LDO5 and LDO6 are examples of a third voltage. The LDO7 to LOD10 and the DC/DC converter are an example of a second voltage regulator, and the voltages generated by the LDO7 to LOD10 and the DC/DC converter is an example of a second voltage. In the following description, LDO1 to LDO10 are also referred to as a LDO when LDO1 to LDO10 is explained without differentiations.

The LDO5 receives +5.5 V generated by the LDO1 through the data generation unit AFE_U, generates a DC voltage of +5 V, and supplies the generated voltage only to a circuit in the amplifier unit AMP_U. The LDO6 receives −6V generated by the LDO2 through the data generation unit AFE_U, generates a DC voltage of −5V, and supplies the generated voltage only to the circuit in the amplifier unit AMP_U. By using a DC voltage generated by the LDO5 and the LDO6 only in the circuit mounted in the amplifier unit AMP_U, it is possible to suppress the power source noise which enters the DC voltage generated by the LDO5 and the LDO6.

The LDO 7 receives +5.5 V generated by the LDO 1, generates a DC voltage of +5 V, and supplies the generated voltage only to the circuit in the data generation unit AFE_U. The LDO 8 receives −6V generated by the LDO 2, generates a DC voltage of −5V, and supplies the generated voltage only to the circuit in data generation unit AFE_U.

The DC/DC converter receives +5V generated by the LDO7 and generates DC voltages of +3.3 V, +2.5 V, and +1.1 V, respectively. The generated DC voltage is supplied only to a logic circuit (digital circuit) such as FPGAs (not illustrated) in the data generation unit AFE_U.

LDO9 receives +16V generated by LDO 3, generates a DC voltage of +15 V, and supplies the generated voltage only to the circuit in the data generation unit AFE_U. The LDO 10 receives −16V generated by the LDO4, generates a DC voltage of −15V, and supplies the generated voltage only to the circuit in the data generation unit AFE_U. By using the DC voltage generated by the LDO7 to the LDO10 and the DC/DC converter only in the circuit installed in the data generation unit AFE_U, it is possible to suppress the power source noise which intrudes into the DC voltage generated by the LDO7 to the LDO10.

In FIG. 3, similarly to FIGS. 1 and 2, a post-regulator is formed in two stages. The first stage is mounted on the backplane unit BP_U, and the second stage is mounted on the board of the data generation unit AFE_U connected to the backplane unit BP_U. In FIG. 3, a second-stage post-regulator is mounted on the amplifier unit AMP_U connected to the backplane unit BP_U through the data generation unit AFE_U.

In the data processing unit FLL_U, the power source voltages +15 V, −15 V, +5 V, and −5 V are used as the power source for the analog circuit. The power source voltages +15 V, −15 V, +5 V, and −5 V are generated by LDO5 to LDO10 mounted on two boards of the data processing unit FLL_U, along with the amplifier 11, the AD converter 12, the DA converter 14, and the like. The reason that the potential difference (0.5 V) between the inputs and outputs of the LDO5 and the LDO7 is smaller than the potential difference (1.0 V) between the inputs and outputs of the LDO6 and LDO8 is because the current supply is large and the power consumption of the LDO is to be reduced.

The amplifier 11 amplifies the output voltage generated by the SQUID in response to the strength of the magnetic field by the magnetic flux through the SQUID and outputs the amplified output voltage to the AD converter 12. The AD converter 12 converts an analog signal amplified by the amplifier 11 into a digital value (voltage value) and outputs the converted digital value to the digital integrator 13. The digital integrator 13 integrates the change in the SQUID's voltage (precisely the amplified voltage output from the amplifier 11) from a working point (or a locking point) which is the starting point of each period of the Φ-V characteristics and outputs the integrated value, which is the integrated voltage value, to the DA converter 14. The digital integrator 13 outputs the integrated voltage value to the data acquisition unit DAQ_U.

The DA converter 14 converts the integrated voltage value (digital value) of the digital integrator 13 to a voltage and outputs the converted voltage to the voltage-to-current converter 15. The voltage-to-current converter 15 converts the voltage received from the DA converter 14 into a current and outputs the converted current to the return coil 16.

The return coil 16 generates a magnetic flux due to the current received from the voltage-to-current converter 15 and feeds the generated magnetic flux back to the SQUID as the return magnetic flux. That is, the return coil 16 generates a magnetic field that the SQUID receives based on the current from the voltage-to-current converter 15. This allows the voltage generated by the SQUID to be maintained near the working point (linear region) of the Φ-V characteristic, and accurately obtains the biomagnetic field signal measured by the SQUID.

The data acquisition unit DAQ_U performs filtering and thinning processing on the digital signal (magnetic field data) received from the digital integrator 13, acquires the magnetic field data corresponding to the signal band indicated in Table 1, and transfers the acquired magnetic field data to the PC. The PC stores the magnetic field data transferred from the data acquisition unit DAQ_U in a hard disk device, etc., calculates the magnetic field waveform and the current waveform using the stored magnetic field data, and displays the calculated waveform on the display device.

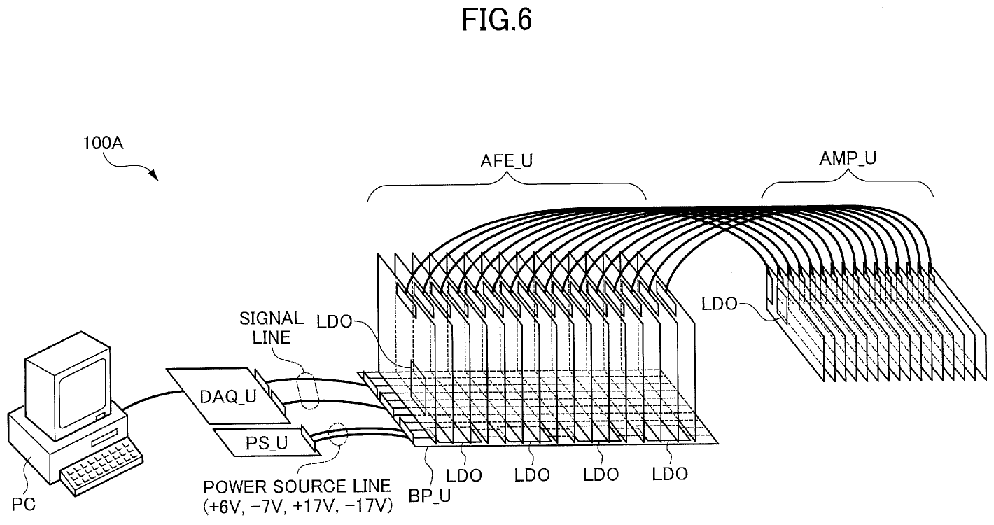

As described in FIGS. 6 to 9, the magnetic field measuring apparatus 100A can operate as the spinal magnetometer, cardio magnetometer, brain magnetometer, or myomagnetometer by replacing the board connected to the backplane unit BP_U. In addition, the magnetic field measuring apparatus 100A can realize both the spinal magnetometer and brain magnetometer at the same time by combining the types of boards connected to the backplane unit BP_U, and can be used with the electroencephalograph, electromyograph, and electrocardiograph.

When a combination of the spinal magnetometer, cardio magnetometer, brain magnetometer, or myomagnetometer is used, the data acquisition unit DAQ_U performs the thinning of the magnetic field data by changing the thinning ratio for each part to be measured.

FIG. 4 is a block diagram illustrating an example of the backplane unit BP_U of FIG. 3. FIG. 4 illustrates an outline of the printed wiring board forming the backplane unit BP_U in a planar view.

The backplane unit BP_U has a power connector PS_CON, a power supply circuit PSC, a plurality of connector slots SLT that are attachably and detachably connected to the printed wiring board and so on, an interface connector DAQIF_CON, and an end connector END_CON. In FIG. 4, the backplane unit BP_U has 16 connector slots SLTs, but the number of the connector slots SLTs is not limited to 16.

The power supply connector PS_CON is connected to the power supply cable that supplies power (power source voltage: +6V, −7V, +17V, and −17V) from the power source unit PS_U. The power source voltage (+6 V, −7 V, +17 V, −17 V) is supplied to the power supply circuit PS through the power connector PS_CON.

The power supply circuit PSC has LDO1, LDO2, LDO3, and LDO4 and outputs the generated power source voltage (+5.5 V, −6 V, +16 V, −16 V) to each connector slot SLT. Although it is not specifically limited, for example, the LDO1 is provided corresponding to each connector slot SLT, the LDO2 is provided corresponding to each of the two connector slots SLT, and the LDO3 and LDO4 are provided common to all connector slots SLT. The number of LDOS to be installed in the backplane unit BP_U is determined by the required current amount for each power source.

The interface connector DAQIF_CON, each connector slot SLT, and the end connector END_CON are connected in sequence through the data interface DIF (data line) and the control interface CIF (control line). The interface connector DAQIF_CON is an example of the second connecting part, and the end connector END_CON is an example of the third connecting part.

The data interface DIF and control interface CIF include the wiring and interface circuit provided on the printed wiring board of the backplane unit BP_U. The data interface DIF and the control interface CIF are connected to the data acquisition unit DAQ_U via a cable connected to the interface connector DAQIF_CON. The data interface DIF and control interface CIF are an example of the common signal line.

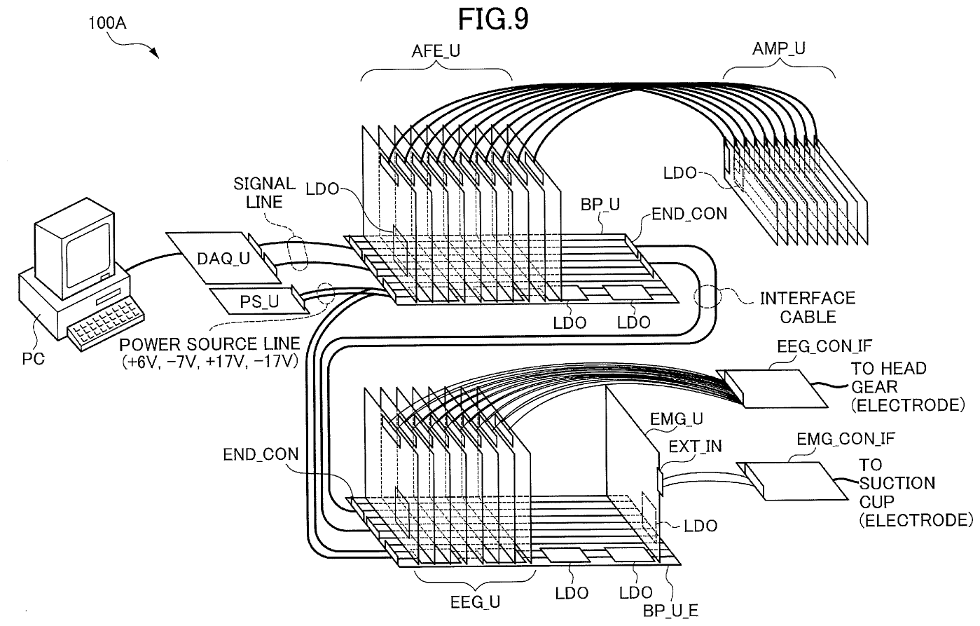

The end connector END_CON is used when connecting the backplane unit BP_U to the expansion backplane unit BP_U_E (FIG. 9). The backplane unit BP_U_E has the same structure as the backplane unit BP_U, and is an example of the fifth board. For example, the end connector END_CON is connected through a cable to the end connector END_CON of the expansion backplane unit BP_U_E. Thus, even when the number of boards connected to the backplane unit BP_U is larger than the number of connector slots SLTs, multiple backplane units BP_U and BP_U_E can be used to connect all boards to the connector slot SLT.

The data interface DIF transfers the magnetic field data generated by the data processing unit FLL_U connected to the connector slot SLT to the data acquisition unit DAQ_U. The control interface CIF transmits a control signal used by the control circuit, etc. connected to the connector slot SLT and the data acquisition unit DAQ_U to each other. For example, a serial interface, such as SerDes (SERIalizer/DESerializer), is used as a data interface DIF and control interface CIF, and a serial signal is transferred. By employing the serial interface such as SerDes, the data can be transmitted at a high speed without considering the skew of the data.

In the magnetic field measuring apparatus 100A, as illustrated in Table 1, the number of channels may be hundreds of channels, and the amount of magnetic field data measured for each channel is also large. Therefore, the data transfer rate of the data interface DIF is set to be two to three digits higher than the data transfer rate of the control interface CIF. Separate wiring of the data interface DIF and the control interface CIF facilitates differences in data transfer rates.

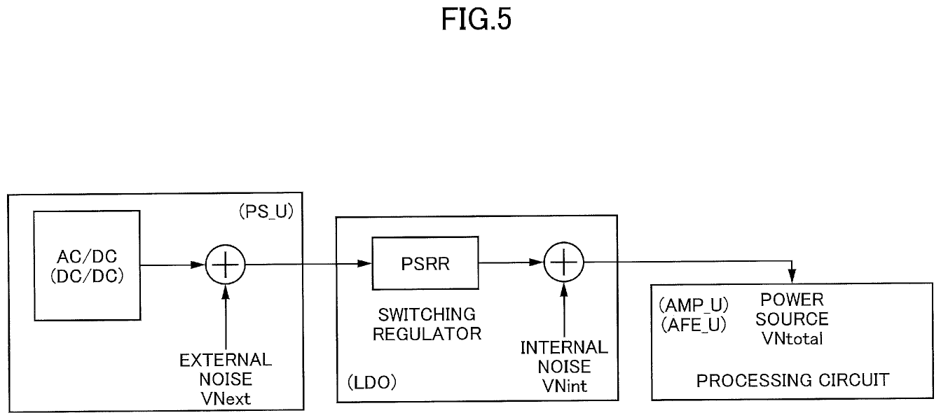

FIG. 5 is a diagram illustrating an example of power source noise generated when using a post-regulator. In FIG. 5, it is assumed that the power source voltage supplied from the supply power source such as the power supply unit PS_U is supplied to the processing circuit such as the amplifier unit AMP_U and data generation unit AFE_U through the voltage regulator such as LDO.

The power source noise (voltage) of the processing circuit shall be VNtotal, voltage noise (external noise) which is generated by the power supply and enters the voltage regulator shall be VNext, and power source noise (internal noise) which is generated by the voltage regulator and enters the processing circuit shall be VNint. The power source voltage variation removal ratio of the voltage regulator is PSRR (Power Supply Rejection Ratio). These relationships are known to be expressed by equation (1) from the definition of PSRR.

[Formula 1]

VNtotal=\sqrt{(VNext×PSRR)²+VNint²} (1)

Because the PSRR value is indicated by a negative decibel value and is taken to be less than 1, the higher the absolute value of the decibel value, the higher the noise elimination ratio and the smaller the effect of external noise VNext on the power source noise VNtotal of the processing circuit.

Also, when the processing circuit is an analog circuit, especially when the first stage is an amplifier (such as the amplifier 11 of FIG. 3), the PSRR of the amplifier itself allows further suppression of the effects of power source noise. In the case of the FLL circuit, Eq. (2) is established when the noise superimposed on the input referred noise of the first stage amplifier is VNadd and the PSRR of the amplifier is PSRRamp (negative value).

[Formula 2]

VNadd=VNtotal×PSRRamp (2)

As represented in Eq. (2), power source noise can be suppressed by the PSRR of the amplifier.

On the other hand, because the input referred noise density Vn of the amplifier itself is about 1 nV/(Hz)1/2, it is very difficult to keep it below this Vn value with a single-stage post-regulator alone. In addition, PSRRs have frequency characteristics, and because the higher the frequency, the smaller the absolute value of the decibel value (the lower a noise rejection ratio), it is even more difficult to keep them below the Vn value considering the higher frequency side PSRR.

In the magnetic field measuring apparatus 100A illustrated in FIG. 3, a circuit susceptible to a noise is the amplifier 11 that amplifies a weak output signal from the SQUID. For example, the amplifier unit AMP_U design including the amplifier 11 performs the switching power supply (AC/DC) with the power supply filter used for the power source unit PS_U as the cutoff frequency=100 kHz and the power supply ripple=10 mVp-p (max). A switching power supply having this performance is available.

The bandwidth of the amplifier 11 mounted in the amplifier unit AMP_U is set to 300 kHz, and the low-pass noise (0.1 Hz-10 Hz) is sufficiently low to be negligible. An input referred noise value EN (Vp-p value) when the input referred noise including the input resistance of the amplifier 11 is 1.0 nVrms/(Hz)1/2 and the Crest Factor (wave height rate) is 6.6 (±3.3σ) is represented in Eq. (3). This amplifier has a PSRR of substantially 100 Hz with a positive power supply of substantially −110 dB, a negative power source of substantially −120 dB and 100 kHz, a positive power supply of substantially −50 dB and a negative power source of substantially −60 dB.

[Formula 3]

EN=0.001×\sqrt{300000}×6.6=3.615 (μVp-p) (3)

In Eq. (3), 0.001 is the value converted from 1.0 nV of input referred noise to μV, 300,000 is the value converted from 300 kHz of the band of amplifier 11 to Hz, and 6.6 is the Crest Factor.

If the effect of power source noise (i.e., Vnext×PSRR) is stricter and the input referred noise value EN of Eq. (3) is about 1/10, it is considered that the effect of the power source noise on the amplifier 11 can be almost ignored. Therefore, in this example, the target value is 0.3615 μVp-p, which is 1/10 of the input referred noise value EN of Eq. (3). The entire input referred noise can be calculated by the square root of square sums.

In the structure illustrated in FIGS. 3 and 4, it is preferable that the first stage LDO1 to LDO4 have a large current capacity because the power source supply amount to the plurality of boards (FLL_U) is not insufficient and the power source current has some margin. On the other hand, the LDO5, the LDO6, and the LDO7 to the LDO10 of the second stage may be provided with the amplifier unit AMP_U and the data generation unit AFE_U, respectively. Generally, a high current capacity voltage regulator tends to have a higher self-noise than low current capacity voltage regulators.

For example, in the +5 V system power supply used in the data processing unit FLL_U, the performance of the first stage LDO1 is as follows: output current=3 A, noise=60 μVrms (10 Hz-100 kHz), PSRR=−60 dB (100 Hz), and −30 dB (100 kHz). The performance of LDO 5 and LDO 7, which are the power sources for the second +5V system, shall be as follows: output current=0.5 A, noise=15 μVrms (10 Hz-100 kHz), PSRR=−60 dB (100 Hz), and −30 dB (100 kHz). Based on this data, a noise value is specifically calculated.

The noise value in the first stage LDO1 is calculated as 506.77 (μVp-p) as indicated in Eq. (4), taking into account the cutoff frequency of the filter of the switching power supply, 100 kHz.

[Formula 4]

\sqrt{(10000×−30 dB)²+(60×6.6)²}=\sqrt{(316.23)²+(396)²}=506.77(μVp-p) (4)

In Eq. (4), 10000 is the conversion of 10 mV of the power source ripple to μV and −30 dB is the PSRR at 100 kHz. 60 is the noise value (60 μVrms) of the first stage LDO1 and 6.6 is the Crest Factor.

When the post-regulator is structured as one stage, the effect of power source noise at the input of the amplifier 11 is 506.77 x −50 dB=1.603 (μVp-p) when the PSRR of the positive power supply at 100 kHz is −50 dB. For this reason, the noise exceeds the target value (0.3615 μVp-p) in the one-stage structure. Said differently, it is difficult to suppress noise.

In the positive power supply (+5 V), the noise value at LDO1 (506.77 (μVp-p)) in Eq. (4) is used, and the noise at the second stage is calculated as 100.29 (μVp-p) as indicated by Eq. (5).

[Formula 5]

\sqrt{(506.77×−30 dB)²+(15×6.6)²}=\sqrt{(16.025)²+(99)²}=100.29(μVp-p) (5)

In Eq. (5), −30 dB is the PSRR at 100 kHz, 15 is the noise value (15 μVrms) of the second stage LDO5, LDO7, and 6.6 is the Crest Factor.

When the post-regulator is structured to have two stages, the effect of power source noise at the input of the amplifier 11 is 100.29×−50 dB=0.317 (μVp-p) when the PSRR of the positive power supply at 100 kHz is −50 dB. In the second stage, the effects of self-noise of LDO are large. However, the target value (0.3615 μVp-p) can be reduced to below 0.3615 μVp-p by using the two-stage structure. Incidentally, the above-described calculation can be performed not only for the positive power supply (+5 V system) but also for the negative power source (−5 V system).

The performance of the LDO2 in the first stage of the −5V system power source is as follows: output current=1.5 A, noise=60 μVrms (10 Hz-100 kHz), PSRR=−62 dB (100 Hz), and −40 dB (100 kHz). The performance of the second stage LDO6 and LDO8 is as follows: output current=0.6 A, noise=18 μVrms (10 Hz-100 kHz), PSRR=−85 dB (100 Hz), and −35 dB (100 kHz). The noise value at the first stage is calculated as 408.43 (μVp-p) as represented in Eq. (6) in the same manner as in Eq. (4).

[Formula 6]

\sqrt{(10000×−40 dB)²+(60×6.6)²}=\sqrt{(100)²+(396)²}=408.43(μVp-p) (6)

In Eq. (6), 10000 is the conversion of 10 mV of the power supply ripple to μV, and −40 dB is the PSRR at 100 kHz. 60 is the noise value (60 μVrms) of the first stage LDO2 and 6.6 is the Crest Factor.

When the post-regulator is structured as one stage, the effect of a noise of a negative power source at an input of the amplifier 11 is 408.43×-60 dB=0.408 (μVp-p) when the PSRR of the negative power source at 100 kHz is −60 dB. This value is better than the positive power source. However, as with the positive power source, the noise exceeds a target value (0.3615 μVp-p) in the one-stage structure.

For the negative power source (−5V), the noise value at the second stage is calculated as 119.02 (μVp-p), as represented in Eq. (7), using the noise value at the LDO2 (408.43 (μVp-p)) in Eq. (6).

[Formula 7]

\sqrt{(408.43×−35 dB)²+(18×6.6)²}=\sqrt{(7.263)²+(118.8)²}=119.02(μVp-p) (7)

In Eq. (7), −35 dB is the PSRR at 100 kHz, 18 is the noise value (18 μVrms) of the second stage LDO6, LDO8, and 6.6 is the Crest Factor.

When the negative power source (−5V) has the two-stage structure of post-regulator, the noise effect of the negative power source at the input of the amplifier 11 is 119.02×−60 dB=0.119 (μVp-p) when the PSRR of the negative power source at 100 kHz is −60 dB. Therefore, the target value can be attained by making the negative power source have two stages.

As described above, the two-stage structure of post-regulator can reduce the influence of noise in the circuit mounted in the data processing unit FLL_U such as the amplifier 11 compared to a one-stage structure of post-regulator. If the performance of the voltage regulator such as LDO improves and the PSRR (absolute value) increases in the future, it is possible that the target value can be cleared even in the one stage structure.

However, when a high-performance LDO is installed in the backplane unit BP_U and the LDO is not installed in the data processing unit FLL_U, the output of the LDO must be wired to the circuit on the data processing unit FLL_U. As a result, the power source noise may intrude through the wiring. When the power source noise enters, the PSRR provided by the amplifier 11 or the like may not sufficiently suppress the power source noise.

If LDO is installed in the data processing unit FLL_U without mounting LDO in the backplane unit BP_U, LDO must be mounted on each board of the amplifier unit AMP_U and the data generation unit AFE_U. Accordingly, more LDO is required than the number of LDOs mounted in the backplane unit BP_U, thereby increasing the cost of the magnetic field measuring apparatus 100A. In other words, by providing the two-stage structure of post-regulator, it is possible to mount a relatively inexpensive LDO on each board of the data processing unit FLL_U, and it is possible to suppress the increase in cost of the magnetic field measuring apparatus 100A.

FIG. 6 is a diagram illustrating an example of a board connected to the backplane unit BP_U in the magnetic field measuring apparatus 100A of FIG. 3. In FIG. 6, the data acquisition unit DAQ_U is connected to the backplane unit BP_U through a cable for the signal line (the data interface DIF and the control interface CIF in FIG. 4). The power source unit PS_U is connected to the backplane unit BP_U through a cable for the power source line.

In FIG. 6, a board of the data generation unit AFE_U is connected to all connector slots SLTs (e.g., 16) of the backplane unit BP_U, and the magnetic field measuring apparatus 100A is used, for example, as a magnetoencephalograph having the SQUID of a 256 channel.

The amplifier unit AMP_U is connected to backplane unit BP_U via data generation unit AFE_U. A SQUID (not shown) and a return coil 16 are connected to the amplifier unit AMP_U. If the circuit cannot be fully mounted in the data generation unit AFE_U, the amplifier unit AMP_U with the remaining circuit is connected to the data generation unit AFE_U. This allows a common connector slot SLT to be used even when a predetermined function is implemented on a plurality of boards.

The power source and signal are supplied to the data generation unit AFE_U through the connector slot SLT of the backplane unit BP_U, which also serves as a housing. In addition, the power is supplied from the data generation unit AFE_U to the amplifier unit AMP_U through a cable.

In FIG. 6, for ease of understanding, the housing is not shown, but the data generation unit AFE_U and the amplifier unit AMP_U are housed in a common or individual housing (e.g., a rack). The backplane unit BP_U is installed in a housing in which the data generation unit AFE_U is housed and integrated with the housing. For example, the data acquisition unit DAQ_U and the power source unit PS_U are housed in an individual housing.

For example, the SQUID, the amplifier unit AMP_U, and data generation unit AFE_U are installed in the magnetic shielding room, and the power source unit PS_U, the data acquisition unit DAQ_U, and PC are installed outside the magnetic shielding room. The data generating unit AFE_U may be installed outside the magnetic shielding room.

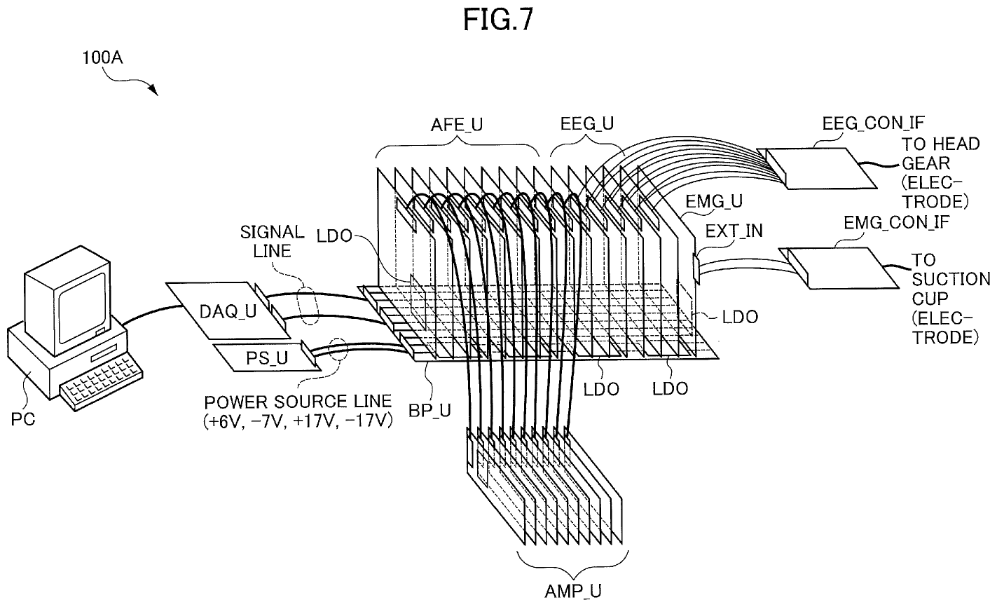

FIG. 7 is a diagram illustrating another example of a board connected to the backplane unit BP_U in the magnetic field measuring apparatus 100A of FIG. 3. For the same elements and structure as in FIG. 6, the detailed description is omitted. The data generation unit AFE_U, the amplifier unit AMP_U, and the SQUID are connected in a manner similar to that described in FIG. 6.

In FIG. 7, 10 (ten) data generation units AFE_U, 5(five) EEG_U, and 1 (one) EMG_U are connected to the connector slot SLT of the backplane unit BP_U. The EEG_U and EMG_U units for respectively measuring electroencephalogram and myoelectric potential have the form of a printed wiring board, for example, and LDO is installed on the printed wiring board. In addition, according to the medical safety standards, the external input is an insulated input, and an insulated regulator is used for the power source on each board. The electroencephalogram measuring unit EEG_U and the myoelectric potential measuring unit EMG_U are examples of a fourth board.

Each electroencephalogram measuring unit EEG_U is connected to the connection interface board EEG_CON_IF and is also connected to an electrode of a head gear of an electroencephalograph through the connection interface board EEG_CON_IF. The function of the electroencephalograph is substantialized by the electroencephalogram measuring unit EEG_U, connection interface board EEG_CON_IF, and the electrode of the head gear of the electroencephalograph.

The myoelectric potential measuring unit EMG_U is connected to the suction cup (electrode) of the electromyograph through the connection interface board EMG_CON_IF. The function of the electromyograph is substantialized by the myoelectric potential measuring unit EMG_U, the connection interface board EMG_CON_IF, and the suction cup (electrode).

In FIG. 7, the magnetic field data measured by the SQUID, the electroencephalographic data measured by the electroencephalograph, and the electromyographic data measured by the electromyograph are transferred to the data acquisition unit DAQ_U through the data interface DIF of the backplane unit BP_U and stored in the PC. The PC displays a waveform etc. on a display device using the stored data. The electroencephalograph and electromyograph are example of a potential measuring instrument. The electroencephalogram measuring unit EEG_U includes a part of the functions of the electroencephalograph, and the myoelectric potential measuring unit EMG_U includes a part of the functions of the electromyograph.

In the example illustrated in FIG. 7, the magnetic field measuring apparatus 100A is used, for example, as a magnetoencephalograph having a SQUID of 160 channels, in synchronization with an electroencephalograph.

Said differently, the magnetic field measuring apparatus 100A illustrated in FIG. 7 functions as a multimodality apparatus using a plurality of modality apparatuses (medical imaging apparatus). Thus, the magnetic field measuring apparatus 100A can be used in conjunction with an electroencephalograph, electromyograph, or electrocardiograph.

To obtain biometric data, the measurement data obtained by the electroencephalograph, electromyograph, or electrocardiograph must be synchronized with the magnetic field data. The electroencephalograph, electromyograph, and electrocardiograph differ in signal size, band, and number of channels for each apparatus, as well as the magnetic field measuring apparatus. For example, by providing the function of the magnetic field measuring apparatus to the electroencephalograph, electromyograph, or electrocardiograph, the synchronization of data is facilitated to make it advantageous in terms of cost and convenience. However, if the power source current increases due to the aggregation of functions and the power source noise increases, the measurement accuracy such as magnetic field data may decrease.

In this embodiment, the LDO connected in series with the LDO mounted in the backplane unit BP_U is installed in a board of each device connected to the backplane unit BP_U. Therefore, the noise included in the power source voltage supplied to the processing circuits of each board can be reduced, and the measurement accuracy of the biological signal can be prevented from being reduced by the magnetic field measuring apparatus 100A which functions as a multimodality apparatus.

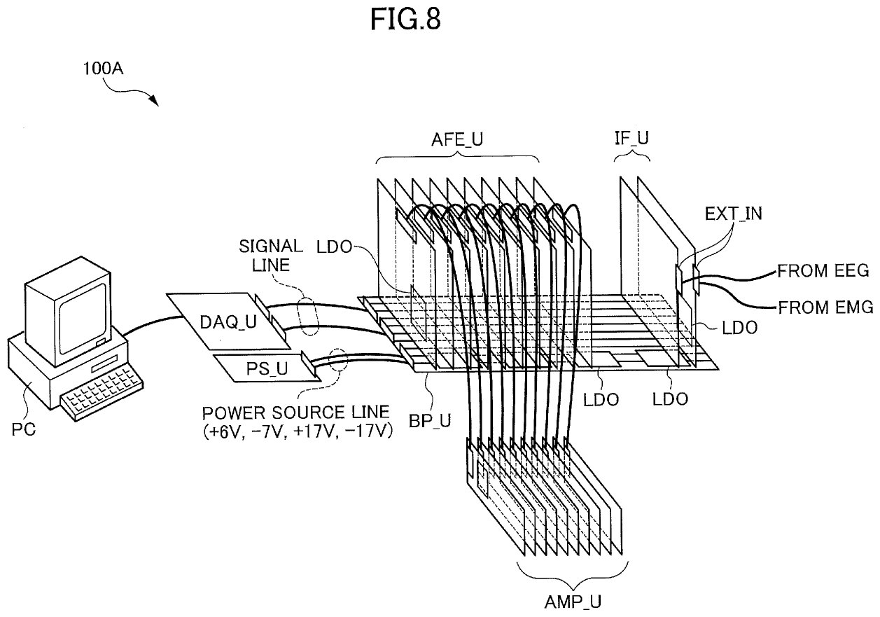

FIG. 8 is a diagram illustrating another example of the board connected to the backplane unit BP_U in the magnetic field measuring apparatus 100A illustrated in FIG. 3. For the same elements and structure as FIGS. 6 and 7, the detailed description is omitted.

FIG. 8 illustrates the difference between the number of data generation units AFE_U connected to the backplane unit BP_U and the structure of the electroencephalograph and the electromyograph. The data generation unit AFE_U connected to the backplane unit BP_U is 10 sheets. The connections of the data generation unit AFE_U and the amplifier unit AMP_U are the same as in FIG. 7.

In FIG. 8, two connector slots SLTs of the backplane unit BP_U are connected to two interface units IF_U (boards) each having an LDO. One of the interface units IF_U is connected to an electroencephalograph (EEG) and another of the interface units IF_U is connected to an electromyometer (EMG). The magnetic field measuring apparatus 100A is used in synchronization with the electroencephalograph and the electromyograph as a magnetoencephalograph having a SQUID of 160 channels, in a manner similar to that of FIG. 7. The interface unit IF_U is an example of a fifth board.

FIG. 9 is an explanatory diagram illustrating yet another example of a board connected to the backplane unit BP_U in the magnetic field measuring apparatus 100A of FIG. 3. For the same elements and structure as these in FIGS. 6 and 7, the detailed description is omitted.

In FIG. 9, the backplane unit BP_U_E for expansion is connected to the backplane unit BP_U. The structure of the backplane unit BP_U_E is the same as that of backplane unit BP_U except that it has an end connector END_CON instead of an interface connector DAQIF_CON. Said differently, the backplane unit BP_U_E has a plurality of connector slots SLT, in which the data interface DIF (data lines) and the control interface CIF (control line) are interconnected, in a manner similar to FIG. 4. The data interface DIF and the control interface CIF are connected to the end connector END_CON attached to both ends of the backplane unit BP_U_E. The backplane unit BP_U_E is an example of the sixth board.

For example, the end connector END_CON of the backplane unit BP_U_E is connected to the end connector END_CON of the backplane unit BP_U by an interface cable. This allows the data interface DIF of the backplane unit BP_U_E and the data interface DIF of the backplane unit BP_U to be connected to each other. The control interface CIF of the backplane unit BP_U_E and the control interface CIF of the backplane unit BP_U are connected to each other.

In the connector slot SLT of the backplane unit BP_U_E, as in a manner similar to FIG. 7, eight (8) electroencephalogram measuring units EEG_U and one (1) myoelectric potential measuring unit EMG_U are connected. The connections of the data generation unit AFE_U, the amplifier unit AMP_U, and the SQUID are the same as in FIG. 7. The functions of the electroencephalograph and electromyograph are substantialized by respectively an electroencephalogram measuring unit EEG_U and myoelectric potential measuring unit EMG_U connected to the backplane unit BP-U-E. EEG data measured by the EEG and myoelectric data measured by the EEG are sequentially transferred to the data acquisition unit DAQ_U through the data interface DIF between the backplane unit BP_U_E and the backplane unit BP_U.

As illustrated in FIGS. 7 and 8, a data generation unit AFE_U for a brain magnetometer and a board for an electroencephalograph and an electromyograph may be connected to the backplane unit BP_U. The backplane unit BP_U_E may be connected with the data generation unit AFE_U for the spinal magnetometer, cardio magnetometer or myomagnetometer. This allows one magnetic field measuring apparatus 100A to function as multiple types of magnetic field measuring apparatuses.

As described above, in the second embodiment, the same effect as the first embodiment can be obtained. For example, by connecting one of the LDO1 to LDO4 and one of the LDO5 to LDO10 in series, the post-regulator is formed as the two-stage structure, thereby reducing the power source noise supplied to the processing circuit mounted in the data processing unit FLL_U or the like.

By mounting the LDO5-LDO 10 in the amplifier unit AMP_U connected to the backplane unit BP_U and the data generation unit AFE_U, noise that enters into the power source voltage generated by the LDO5-LDO10 can be reduced. When designing a new circuit board that connects to the connector slot SLT, the appropriate LDO (second stage) can also be selected for the processing circuit mounted on the circuit board. Accordingly, by combining the first stage LDO1 to LDO4 and the second stage LDO, the amount of noise reduction can be set to a desired amount, thereby preventing the measurement accuracy of the magnetic field measuring apparatus 100A from being deteriorated.

By replacing the board connected to the backplane unit BP_U, it can be operated as a spinal magnetometer, cardio magnetometer, brain magnetometer, or myomagnetometer. In addition, the magnetic field measuring apparatus 100A can substantiate both the spinal magnetometer and brain magnetometer at the same time by combining the types of boards connected to the backplane unit BP_U, and can be used in combination with the electroencephalograph, an electromyograph, and an electrocardiograph. As a result, for example, it is possible to place the magnetic field measuring apparatus 100A having a plurality of functions in one magnetic shield room, thereby significantly reducing the total cost of the magnetic field measuring system.

Further, in the second embodiment, if the circuit cannot be sufficiently installed on the second board (AFE_U) connected to the connector slot SLT, the remaining circuit can be mounted on the third board (AMP_U). This allows a common connector slot SLT to be used even when a predetermined function is implemented by a plurality of boards. In this case, the power source supplied from the first board (BP_U) to the second board is available for the power source to the LDO (LDO5, LDO6) installed on the third board.

By using the DC voltage generated by the LDO7 to the LDO10 and the DC/DC converter only in the circuit installed in the data generation unit AFE_U, it is possible to suppress the power source noise which enters the DC voltage generated by the LDO7 to the LDO10. By using the DC voltage generated by the LDO5 and the LDO6 only in the circuit mounted in the amplifier unit AMP_U, it is possible to suppress the power source noise which enters the DC voltage generated by the LDO5 and the LDO6.

By providing an end connector END_CON for the backplane unit BP_U, the backplane unit BP_U for expansion can be connected to the backplane unit BP_U. Thus, even when the number of boards connected to the backplane unit BP_U is larger than the number of connector slots SLTs, multiple backplane units BP_U and BP_U_E can be used to connect all boards to the connector slot SLT.

By wiring the data interface DIF and the control interface CIF separately on the backplane units BP_U and BP_U_E, the data transfer rate can be easily differed. By employing a serial interface such as SerDes, for example, data can be transmitted at high speed without considering skew.

### Third Embodiment

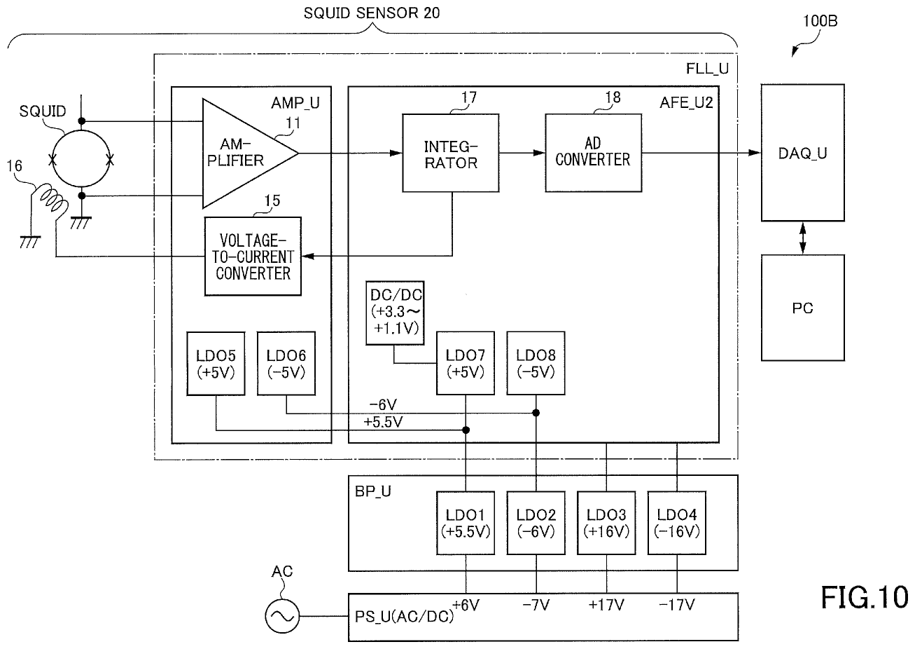

FIG. 10 is a block diagram illustrating an example of a magnetic field measuring apparatus according to a third embodiment of the present invention. For the same structure as illustrated in FIG. 3, the same reference numerals are given and a detailed description thereof is omitted. The magnetic field measuring apparatus 100B illustrated in FIG. 10 employs an analog FLL method, and the data processing unit FLL_U has a data generation unit AFE_U2 instead of the data generation unit AFE_U illustrated in FIG. 3. Other configurations of the magnetic field measuring apparatus 100B are similar to those of the magnetic field measuring apparatus 100A illustrated in FIG. 3.

The data generation unit AFE_U2 includes an LDO7, an LDO8, a DC/DC converter, an integrator 17, and an AD converter 18. The two-stage structure of the LDO that is the post-regulator is the same as in FIG. 3, except that the LDO9 and the LDO10 are not present.

The integrator 17 is an analog circuit and has the same function as the digital integrator 13 of FIG. 3. The integrator 17 integrates the change in voltage of the SQUID from the working point of the Φ-V characteristic and outputs the integrated voltage (signal voltage) to the voltage-to-current converter 15 and the AD converter 18. The AD converter 18 converts the voltage from the integrator 17 to a digital value and outputs it to a data acquisition unit DAQ_U.

As described above, in the third embodiment, the same effect as in the first embodiment and the second embodiment can be obtained. That is, in the magnetic field measuring apparatus 100B of the analog FLL method, the power source noise supplied to the processing circuit in the data processing unit FLL_U can be reduced by making the post-regulator have the two-stage structure.

Although the above-described embodiment has been described as being applied to a biomagnetic field measuring apparatus such as the brain magnetometer, spinal magnetometer, and cardio magnetometer, it may be applied to a magnetic field measuring apparatus other than a biomagnetic field measuring apparatus.

### Effects of the Invention

The noise contained in the second voltage generated based on the power supplied from the power supply unit can be reduced to improve the accuracy of the measurement.

### DESCRIPTION OF SYMBOLS

20: SQUID sensor
30, 30A: Digital FLL circuit
11: Amplifier
12: AD converter
13: Digital integrator
14: D-A converter
15: Voltage-to-current converter
16: Return coil
17: Integrator
18: AD converter
100, 100A, 100B: Magnetic field measuring apparatus
AC: AC power source
AFE_U, AFE_U2: Data generation units
AMP_U: Amplifier unit
BP_U, BP_U_E: Backplane unit
CB1: Cable
CIF: Control interface
CN1: Connector
DAQIF_CON: Interface connector
DAQ_U: Data acquisition unit
DIF: Data interface
EEG_CON_IF: Connection interface board
EEG_U: Electroencephalogram measuring unit
EMG_CON_IF: Connection interface board
EMG_U: Myoelectric potential measuring unit
END_CON: End connector
FLL_U: Data Processing Unit
IFU: Interface Unit
PSC: Power supply circuit
PS_CON: Power connector
PS_U: Power source unit
SLT: Connector slot
TL: Transmission line
VR1, VR2, VR3, VR4, VR5: Voltage regulator

Although the invention has been described in accordance with the embodiments, the invention is not limited to the requirements illustrated in the embodiments. In these respects, the subject matter of the present invention may be varied without prejudice and may be suitably defined according to its application.

All examples and conditional language recited herein are intended for pedagogical purposes to aid the reader in understanding the principles of the invention and the concepts contributed by the inventor to furthering the art, and are to be construed as being without limitation to such specifically recited examples and conditions, nor does the organization of such examples in the specification relate to a showing of the superiority or inferiority of the invention. Although a magnetic field measuring apparatus has been described in detail, it should be understood that various changes, substitutions, and alterations could be made thereto without departing from the spirit and scope of the invention.

The order of the method of the embodiment of the present invention is not limited to the order of processes of the method disclosed by this disclosure.

The present invention can be implemented in any convenient form, for example using dedicated hardware, or a mixture of dedicated hardware and software. The present invention may be implemented as computer software implemented by one or more networked processing apparatuses. The network can comprise any conventional terrestrial or wireless communications network, such as the Internet. The processing apparatuses can compromise any suitably programmed apparatuses such as a general purpose computer, personal digital assistant, mobile telephone (such as a WAP or 3G-compliant phone) and so on. Since the present invention can be implemented as software, each and every aspect of the present invention thus encompasses computer software implementable on a programmable device. The computer software can be provided to the programmable device using any storage medium for storing processor readable code such as a floppy disk, hard disk, CD ROM, magnetic tape device or solid state memory device.

The hardware platform includes any desired kind of hardware resources including, for example, a central processing unit (CPU), a random access memory (RAM), and a hard disk drive (HDD). The CPU may be implemented by any desired kind of any desired number of processor. The RAM may be implemented by any desired kind of volatile or non-volatile memory. The HDD may be implemented by any desired kind of non-volatile memory capable of storing a large amount of data. The hardware resources may additionally include an input device, an output device, or a network device, depending on the type of the apparatus. Alternatively, the HDD may be provided outside of the apparatus as long as the HDD is accessible. In this example, the CPU, such as a cache memory of the CPU, and the RAM may function as a physical memory or a primary memory of the apparatus, while the HDD may function as a secondary memory of the apparatus.

What is claimed is:

1. A magnetic field measuring apparatus that includes
a first board having a plurality of first connecting parts,
at least one second board connected to the plurality of first connection parts, and
a power supply unit supplying power to the first board and the at least one second board so as to measure a magnetic field, the magnetic field measuring apparatus comprising:
at least one first voltage regulator disposed on the first board to generate a first voltage using power from the power supply unit; and
at least one second voltage regulator disposed on any one of the at least one second board to generate a second voltage using the first voltage.

2. The magnetic field measuring apparatus according to claim 1,
wherein the plurality of first boards are respectively backplanes,
wherein the at least one second board is attachably and detachably connected to any one of the plurality of first connecting parts of the backplanes.

3. The magnetic field measuring apparatus according to claim 1, the magnetic field measuring apparatus further comprising:
a plurality of FLL circuits each of which includes
an amplifier for amplifying a voltage output from a superconducting quantum interference device in conformity with a change in a magnetic field,
an integrator for integrating a voltage value output by the amplifier,
a voltage-to-current converter for converting the voltage value output by the integrator into a current, and
a coil for generating a magnetic field received by the superconducting quantum interference device based on a current output by the voltage-to-current converter, the integrator being installed in every one of the at least one second board, the second voltage regulator generating the second voltage for use in a circuit installed in the at least one second board; and
a third board connected to the at least one second board, in which the amplifier and the voltage-to-current converter are installed, the third board including a third voltage regulator that generates a third voltage for use in a circuit installed in the third board using the first voltage supplied through the at least one second board.

4. The magnetic field measuring apparatus according to claim 3, the magnetic field measuring apparatus further comprising:
the plurality of second voltage regulators for generating a plurality of mutually different voltages for use in the circuit installed in the at least one second board; and
a plurality of third voltage regulators for generating a plurality of mutually different voltages for use in a circuit installed in the third board.

5. The magnetic field measuring apparatus according to claim 1,
wherein the plurality of first connecting portions is capable of connecting a fourth board including a part of a function of a potential measuring device or a fifth board connected to the potential measuring device.

6. The magnetic field measuring apparatus according to claim 1,
wherein the first board includes
a second connecting part, to which a data acquisition unit for acquiring measured data processed by the circuit installed in the second board is connected, and
a common signal line provided in the plurality of first connecting parts in common and connecting the plurality of first connecting parts to the second connecting part.

7. The magnetic field measuring apparatus according to claim 6,
wherein the common signal line includes a data line through which the measured data is transmitted, and a control line through which a control signal for controlling a circuit installed in the second board is transmitted.

8. The magnetic field measuring apparatus according to claim 7,
wherein the measured data and the control signal are subjected with a serial transmission on the first board.

9. The magnetic field measuring apparatus according to claim 1,
wherein the first board has a third connecting part capable of connecting a sixth board which has the plurality of first connecting part capable of connecting the second board.
